(19) United States
(12) Patent Application Publication
(10) Pub. No.: US 2022/0256880 A1
(43) Pub. Date: Aug. 18, 2022

(54) HIGH QUALITY LYOPHILIZED COFFEE AND METHOD FOR PREPARING SAME

(71) Applicant: GENIALIS, Henrichemon (FR)

(72) Inventors: Isabelle Desjardins-Lavisse, Henrichemont (FR); Guillaume Gillet, Salbris (FR)

(21) Appl. No.: 17/597,376

(22) PCT Filed: Jul. 6, 2020
(86) PCT No.: PCT/FR2020/051198

§ 371 Date: Jan. 4, 2022

(30) Foreign Application Priority Data
Jul. 5, 2019 (FR) FR1907563

(51) Int. Cl. A23F 5/32; A23F 5/30; A23F 5/38; A23F 5/40; A61K 8/9789; A61Q 19/00

(52) U.S. Cl. CPC A23F 5/32; A23F 5/30; A23F 5/38; A61K 2800/10; A61K 8/9789; A61Q 19/00; A61K 2800/84; A23F 5/40

(57) ABSTRACT

A soluble, lyophilized coffee has improved properties compared to known lyophilized coffee and is obtained by a dehydration process implementing a cryogenic step under pressure followed by a lyophilization step.

### CROSS-REFERENCE TO RELATED APPLICATIONS

This application is a national phase entry under 35 U.S.C. § 371 of International Patent Application PCT/FR2020/051198, filed Jul. 6, 2020, designating the United States of America and published as International Patent Publication WO 2021/005300 A1 on Jan. 14, 2021, which claims the benefit under Article 8 of the Patent Cooperation Treaty to French Patent Application Serial No. FR1907563, filed Jul. 5, 2019.

### TECHNICAL FIELD

The present disclosure relates to the field of soluble coffee. More particularly, it relates to a lyophilized coffee exhibiting improved properties compared to the lyophilized coffee of the prior art. This coffee is obtained by a dehydration method implementing a cryogenic step under pressure of a matrix containing dissolved gas followed by a lyophilization step.

### BACKGROUND

Soluble coffee is a powdered coffee used to prepare a drink from coffee beans by lyophilization or atomization. Consumers appreciate it for its speed of preparation.

There are many soluble coffees available on the market. Certain methods for preparing these coffees are the subject of patent protection.

By way of example, French Patent No. FR2524267 describes a method for manufacturing lyophilized coffee having the appearance of roasted and ground coffee. This method comprises a step of slow freezing the lower part of a layer of aqueous coffee extract by leaving the upper part in the state substantially but not completely solidified by freezing; then in instant freezing the upper part of the layer of coffee extract by spraying a cryogenic fluid on its surface; and finally in grinding the frozen coffee extract obtained in the previous step and lyophilizing the frozen and ground coffee extract.

International Patent Application Publication No. W02019058351 describes a lyophilized coffee preparation comprising a dry concentrate and ground coffee particles, the ground coffee particles having a median size that is not greater than nineteen microns and the particles being ground in an aqueous medium. The present disclosure also relates to a preparation in which the roasted ground coffee particles constitute between 3 and 30% of the lyophilized coffee granule.

European Patent Application Publication No. EP2100514A1 relates to a method for preparing a granular instant foaming coffee composition comprising particles having an apparent density of 0.16 to 0.45 g/cm³, the particles comprising a continuous phase comprising an instant coffee matrix and a non-continuous phase comprising particles of a foaming component containing a gas. The method comprises the steps of using a coffee extract in which a gas is directly injected that is mixed with the extract, adding a foaming component to the mixture to form a coffee mixture, freezing the coffee mixture and then lyophilizing the coffee mixture. The obtained compound is then sieved to separate particles of less than 500 micrometers.

International Patent Application Publication No. WO94/28736A1 discloses a low-density heat soluble extractable food product comprising granules, specifically lyophilized tea or coffee granules, and a method for preparing a heat-soluble extractable food product comprising: (a) extracting an extractable food product, (b) cooling the extract and aerating it with a gas comprising: (i) a gas or mixture of gases substantially more soluble than nitrogen; or (ii) a mixture of nitrogen and a gas or mixture of gases substantially more soluble than nitrogen, to produce a partially frozen foam, (c) substantially freezing the foam, (d) breaking the substantially frozen foam into granules, and (e) lyophilizing the granules to yield the heat soluble product.

U.S. Pat. No. 3,575,060 relates to lyophilized coffee extracts and methods and apparatuses for the production thereof. The method involves preparing a coffee concentrate that is then instantly shock frozen by being discharged into a spray nozzle in drops that pass over a low-temperature area created by a surrounding jet of liquid cryogenic gas. The last step is preferably carried out in a free fall system at atmospheric pressure. The frozen drop particles fall by gravity and are collected, after which they are lyophilized to remove moisture content by sublimation.

French Patent No. FR2536961 discloses a method making it possible to obtain a flavor for a food powder. This method comprises a step of spraying an aqueous solution in a fluid at very low temperature such as liquid nitrogen, then lyophilizing the frozen products. The obtained products are microporous (pore radius probably less than 150, or even 50 Angstroms), and are used as a support for trapping aromatic products. The aqueous solution can be an aqueous coffee extract and the aromatic product a coffee oil enriched with grinding gas.

U.S. Pat. No. 3,443,963A relates to improving the color of lyophilized coffee. More particularly, this disclosure relates to controlling the rate of freezing of roasted coffee extract during a small portion of the freezing curve. The method comprises cooling an aqueous extract of coffee from 25 to 10 F over a period of at least 10 minutes to thereby develop large crystals of water from the ice, further cooling the extract below its eutectic point, then lyophilizing the frozen extract.

Thus, the documents of the state of the art describe the use of gas and, in particular, of liquid nitrogen to foam the matrix, that is to say, to make it porous before freezing. Furthermore, the addition of flavor is used to improve the organoleptic properties of lyophilized coffee.

Most of the known methods have been established for many years and few innovations have yet seen the light of day in this field.

To date, lyophilized coffees are not fully satisfactory for freshly brewed coffee lovers, who deplore the altered coffee taste and sometimes difficult solubilization. It is clear that the quality of the native coffee is altered by the freezing and dehydration methods that are currently available.

There is a real need to have a soluble coffee that is similar in perceived quality to a filter coffee or espresso.

### BRIEF SUMMARY

The present disclosure aims to solve this problem by providing a lyophilized coffee exhibiting improved characteristics both as regards the quality of the coffee powder and its organoleptic properties.

The present disclosure relates to a method for preparing a lyophilized coffee comprising:
a) providing a coffee preparation in the form of a liquid, semi-liquid or pasty matrix;
b) dissolving an inert gas in the matrix by passing through a zone dense in gas molecules, such a density being obtained (i) either by the gas flow generated by the evaporation of a cryogenic fluid, (ii) or by an elevation of the pressure, (iii) or by the combination of these two means;
c) cryogenizing the gas-rich matrix obtained in step b) at a pressure allowing the dissolved gas to be maintained in order to obtain frozen granules, particles or beads;
d) lyophilizing the granules, particles or beads; and
e) obtaining the dehydrated coffee in powder form.

The present disclosure also relates to a lyophilized coffee obtained by such a method, as well as to a composition comprising such a coffee and its use for the preparation of drinks and food or cosmetic preparations

The present disclosure aims to remedy the existing problems by proposing a method for obtaining a high-quality lyophilized coffee.

This method comprises dissolving a large quantity of gas in a coffee preparation and then in freezing the obtained matrix via a deep-freezing method under pressure, so as to keep the gas dissolved in the matrix. The frozen matrix is then lyophilized.

The cryogenic step under pressure allows substantial improvements both in terms of drying (during lyophilization) and of the properties of the products produced.

First, the preparation time is much shorter. Cryogenics under pressure is an almost instantaneous method, making it possible to produce balls of initially fluid product continuously and at high rates (several hundred kg per hour on standard equipment). The time saving is considerable compared to freezing in a cold room, even if they operate at very low temperatures (−40° C. to −80° C. in general). The cryogenized beads are extracted from the materials producing them at temperatures generally between −80° C. and −120° C., which makes it possible to start lyophilization directly, with products whose temperature is around −60° C., without a prior cooling step.

More surprisingly, the lyophilization time itself is very greatly reduced (down to a factor of at least 2). The duration of the method as a whole is significantly reduced.

As regards the quality of the lyophilized coffee, using the method according to the present disclosure makes it possible to obtain products in the form of high-quality powder, without the “edge/core effect” usually obtained when the lyophilization is carried out on product plates, which induces a drying gradient and damaged matrices on the exteriors of the plates (usually called “cake”). On the contrary, the spherical shape of the frozen matrix and the presence of gas allow homogeneous dehydration without alteration; the product is therefore of high quality.

Cryogenics under pressure makes it possible to obtain frozen non-porous products containing a large quantity of dissolved gas. As this gas is not oxygen, oxidation reactions are avoided and the quality of the product is therefore preserved. In fact, the inert gases, once dissolved in the matrix, protect the integrity of the structures and preserve the properties of the matrices, in particular, the very specific and intense organoleptic properties of coffee.

In addition, lyophilizing such products makes it possible to eliminate the majority of the water contained in the product. Thus, very surprisingly, the lyophilized coffee powders obtained from cryogenized intermediate products containing dissolved gas are also much more porous than those obtained from conventionally frozen products. Thus, the quality of the dehydrated products is higher than that of the lyophilized products obtained by a conventional method because the conditions used are on the whole gentler, less aggressive, and less destructuring for the coffee material.

Thus, regardless of the pressure applied, the pressurized cryogenized beads containing dissolved gas make it possible to obtain a fine powder, which can be handled simply because it is able to be measured and is not sticky.

Here, the lyophilized coffee powders are very fine, are of very low apparent density and do not require grinding before use. If necessary, they can be compacted for ease of use.

Another advantage of the lyophilized coffee powders according to the present disclosure is that they dissolve quickly, even at room temperature, and do not leave a deposit.

Finally, the method according to the present disclosure and the equipment used allow improved preservation of the integrity of the starting matrix, in particular, in terms of its physicochemical properties. Very advantageously, their organoleptic properties are significantly improved compared to lyophilized coffees obtained by the methods of the prior art.

### BRIEF DESCRIPTION OF THE DRAWINGS

The present disclosure will be better understood upon reading the detailed description of example embodiments that follow, which are provided by way of illustration and should in no case be considered as limiting the scope of the present invention.

FIG. 1: Represents the lyophilization kinetics observed for the 3 preparation conditions. Circle: Reference; Triangle: Cryo LP; Square: Cryo MP.

FIG. 2: Microscopic observations made on the three coffee powders according to the preparation conditions; A: lyophilized coffee particles obtained under Cryo LP condition; B: lyophilized coffee particles obtained under Cryo MP condition; C: lyophilized coffee particles obtained under conventional conditions (reference freezer −20° C.).

FIG. 3: Correlation between density and rehydration time of coffee powders.

FIG. 4: Chromatograms obtained following analysis by an electronic nose of the three powders obtained after lyophilization (Reference conditions, Cryo LP and Cryo MP). In graph 4B: the first peak marked with an arrow comes out at 34.73, corresponds to 2.5 dimethylpyrazine, IP=70.99; the second peak marked with an arrow comes out at 39.29, corresponds to trimethylpyrazine, IP=48.51; the third peak marked with an arrow comes out at 45.66, corresponds to (E, Z) -2.6-nonadienal, IP=35.36.

FIG. 5: PCA analysis on the olfactory profiles of the 3 coffees: Frozen reference; Cryo LP and Cryo MP.

FIG. 6: Correlation between densities and luminances of 3 lyophilized coffee powders.

FIG. 7: Appearance of a food preparation comprising custard and lyophilized coffee obtained under Cryo LP conditions: A: before mixing; B: after light mixing; C: after complete mixing.

FIG. 8: Schematic diagram of the method used showing the effects of pressure and gas dissolution/release in the product, a. preparation of the product at atmospheric pressure; b. gas dissolution in the product, partial pressure Pp of the dissolved gas being the sum of the pressure of the enclosure Pel and the local pressure linked to a gas flow Pfl; c. cryogenics of the product containing the dissolved gas, operating at a pressure Pc greater than or equal to partial pressure Pp of the gas contained in the product, the pressure Pc itself being able to result from the combination of a chamber pressure Pet and the local pressure linked to a gas flow Pf2; d. possible storage of the product in the form of solid beads at atmospheric pressure and at a temperature below the melting point of the product; e. lyophilization under partial vacuum, resulting in the sublimation of the water contained in the product and the release of the gas trapped therein, causing the creation of a microporous structure in the solid product beads; f. storage of the powder obtained at the end of lyophilization, after any crushing of the dry microporous beads.

The arrows represent the gas that is applied to the surface of the product in step b., which remains in equilibrium in step c. and which escapes in step e.

### DETAILED DESCRIPTION

A first object of the present disclosure relates to a method for preparing a lyophilized coffee comprising:
a) providing a coffee preparation in the form of a liquid, semi-liquid or pasty matrix;
b) dissolving an inert gas in the matrix by passing through a zone dense in gas molecules, such a density being obtained (i) either by the gas flow generated by the evaporation of a cryogenic fluid, (ii) or by an elevation of the pressure, (iii) or by the combination of these two means;
c) cryogenizing the gas-rich matrix obtained in step b) at a pressure allowing the dissolved gas to be maintained in order to obtain frozen granules, particles or beads;
d) lyophilizing the granules, particles or beads; and
e) obtaining the dehydrated coffee in powder form.

The dissolved gas is an inert gas such as nitrogen, argon or helium, etc. Using an inert gas makes it possible to avoid oxidation of the matrix, and therefore to preserve the physicochemical and organoleptic properties of the starting matrix.

Within the meaning of the present disclosure, “zone dense in gas molecules” or “zone dense in molecules” means a zone in which the number of molecules per unit of volume is higher than that which would be observed at atmospheric pressure. Since this volume is not closed and can constitute, in particular, a sub-unit of a larger volume, the high number of molecules found there does not necessarily translate into a visible increase in the pressure of the assembly. It is also possible to consider it as a local but non-measurable pressure, the latter being constituted by the combination of the possible pressure applied to the assembly and the effect induced by the density of molecules generated.

When the zone dense in molecules is obtained owing to a gas flow generated by the evaporation of a cryogenic fluid, the quantity of gas dissolved in the matrix depends on the control of the flow and is typically equivalent, when no pressure is applied, to that which would be obtained by the application of relative pressures between 0.001 bars and 2 bars. A particularly preferred cryogenic fluid is liquid nitrogen.

When the zone dense in molecules is obtained by increasing the pressure, this pressure is greater than atmospheric pressure, and may be, in particular, greater than 0.5 bar, 1 bar, 2 bars, 5 bars, 10 bars, 50 bars, 100 bars, 200 bars, even 250 bars or more. In a particular embodiment, it is between 2 and 100 bars.

In a preferred embodiment, the zone dense in molecules is obtained at least in part by a gas flow generated by the evaporation of a cryogenic fluid. It can be obtained by combining the evaporation of a cryogenic fluid with an increase in pressure; this condition corresponds to the combination of the two methods of dissolving the gas in the matrix mentioned in (iii) of step b) of the method.

Within the meaning of the present disclosure, “under pressure” means conditions that allow the dissolution of a gas in a matrix and/or the maintenance of the gas dissolved in the matrix during deep freezing. Pressurization can be obtained either by increasing the pressure or by bringing the matrix into contact with a cryogenic fluid, the evaporation of this gas creating a gas molecule density equivalent to pressurization so that the gas molecules dissolve in the matrix. The increase in pressure can also be obtained by a movement of gas creating a local pressure. In addition, putting “under pressure” corresponds to the application of relative pressures, that is to say, atmospheric pressure is considered to be a pressure of 0 bar. All the pressures expressed in this document are relative pressures, and the method is not carried out under partial vacuum.

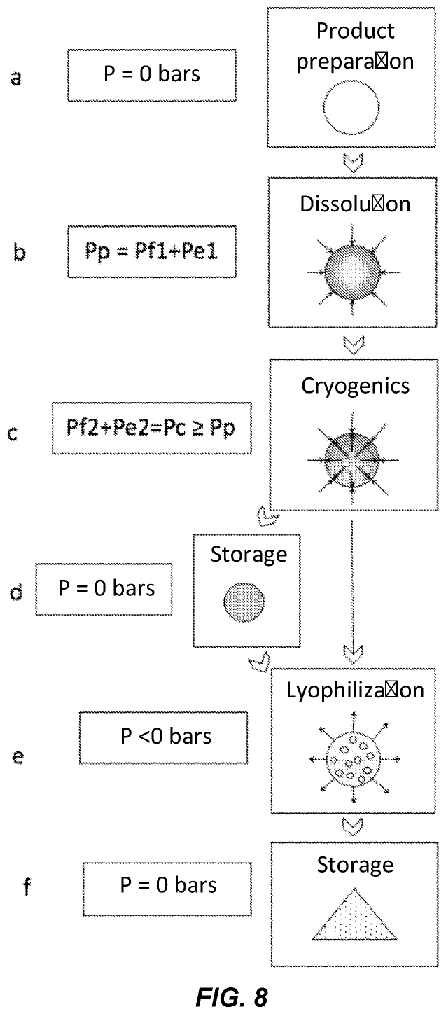

FIG. 8 shows an implementation diagram of the present method in which the zone dense in gas molecules is generated by the combination of a flow of a gas and pressurization of an enclosure.

Thus, step b) takes place at a relative pressure sufficient to allow gas to dissolve in the matrix, and an equivalent pressure is maintained in step c) to keep the dissolved gas inside the matrix during cryonics.

As regards the implementation of the method as a whole, it is possible to chain the stages of the method one after the other and, in particular, to carry out the lyophilization stage immediately after the cryogenics stage. In addition, the method can be carried out continuously. It is also possible to keep the product in frozen form at the end of step c) and to carry out the lyophilization subsequently, after a negative cold storage time to keep the products in the solid state (for example, at −20° C.). In both cases, the advantage of the method is retained.

Thus, according to alternative embodiments, the lyophilization step d) can be carried out either immediately following the cryogenic step c), or subsequently after storage of the frozen granules, particles or beads.

The conditions of the method can be adapted according to the shape of the coffee matrix to be dehydrated, in particular, the pressure during the cryogenic step, and the lyophilization parameters. Those skilled in the art will know how to make such adaptations.

The coffee preparation to be dehydrated can be any type of liquid, semi-liquid or pasty coffee matrix. It is preferably a liquid matrix, comprising a single coffee or a mixture of different coffees when it is desired to mix the flavors, for example.

A second object of the present disclosure relates to a lyophilized coffee capable of being obtained by the method as defined above.

The lyophilized coffee according to the present disclosure is obtained by the method described above. Combining deep freezing under pressure of a coffee matrix containing a dissolved inert gas and lyophilization imparts new properties to the dehydrated coffee powder thus obtained. The properties of this lyophilized coffee result directly from the implemented method and are intrinsically linked to it.

Remarkably, the coffee according to the present disclosure is characterized by the presence of spherical particles. Spherical particles represent a significant fraction of these particles. Thus, the spherical particles represent at least 25% of the powder, or even 30%, 40%, 50%, 60%, 75% or more. This characteristic differentiates this lyophilized coffee from those of the prior art.

Another advantageous property is the fineness of the coffee powder, which is linked to a particle size of less than 30 microns without grinding the powder. In a particularly preferred embodiment, the size of the particles is less than 20 microns. In a very particular embodiment, it is less than 10 microns (still without grinding the powder). These sizes are typically determined by optical microscopy.

In a particular embodiment, the particles of the lyophilized coffee powder can therefore be characterized by the presence of spherical particles, the size of which is less than 30 microns.

This lyophilized coffee also has other properties that differentiate it from lyophilized coffees by a conventional deep-freezing-lyophilization method. Indeed, it is less dense and dissolves more easily. The rate of solubilization is remarkably increased compared to an equivalent coffee obtained by a conventional deep-freezing-lyophilization method, in particular, at room temperature. The coffee also has a color different from that of a coffee obtained by a conventional method; overall, it is brighter and lighter in color. All these characteristics testify to a preparation that is more respectful of the raw material, and has gentler and less destructuring conditions.

In addition, the method according to the present disclosure makes it possible to prepare a lyophilized coffee with significantly improved organoleptic properties compared to an equivalent coffee obtained by a conventional deep-freezing-lyophilization method.

It should be noted that although the coffee thus lyophilized is characterized by a low density, it can be compacted to facilitate its use (moderate compaction to reduce the volume of the packaging, etc.)

Thus, the present disclosure also relates to a lyophilized coffee in compacted form so as to reduce the volume of the product.

A third subject of the present disclosure relates to a composition comprising a lyophilized coffee as defined above.

Such a composition can consist of a mixture of different lyophilized coffees. It can also comprise at least one other ingredient, lyophilized or not, chosen from milk, chocolate, chicory, flavors, carriers (maltodextrin, for example), etc.

A fourth subject of the present disclosure relates to the use of a lyophilized coffee according to the present disclosure for the preparation of drinks or food or cosmetic preparations.

In the food sector, coffee is very popular for its flavors.

In cosmetics, coffee and, in particular, the caffeine it contains, are used for their multiple draining, calming, anti-inflammatory, antioxidant, anti-cellulite, etc., properties.

### EXAMPLES

### Example 1: Implementation of the Method According to the Present Disclosure

1.1 General description of the method

The method according to the present disclosure comprises dissolving a large quantity of a gas in a matrix, in cryogenizing the matrix in the form of granules or beads and then in lyophilizing the matrix.

The gas dissolution and cryogenics steps can be carried out in two distinct ways:
either by incorporating gas at a pressure greater than atmospheric pressure, then by carrying out rapid freezing using cryogenic fluid, as described in application WO2008/043909;
or by incorporating gas by immersion in a cryogenic fluid; in this embodiment, the step of gasifying the matrix comprises dissolving a large quantity of the gas generated by the evaporation of a cryogenic fluid in a matrix so that the product is at least saturated with the gas, the dissolution being carried out by increasing the number of gas molecules in a zone of high gas density, called a “high molecular density zone,” located above the surface of the cryogenic fluid and on the path of the matrix drops before their immersion in the fluid, the high molecular density zone being created by carrying out the gasification and cryonics of the gasified matrix within a closed chamber provided with a vent arranged to allow evacuation of the gas generated by the evaporation of the cryogenic fluid by natural convection and to keep the interior of the enclosure at a pressure greater than or equal to atmospheric pressure;
or by incorporating gas by immersion in a cryogenic fluid while applying a pressure greater than atmospheric pressure.

The matrices rich in gas and in the form of frozen granules, particles or beads are then subjected to lyophilization according to conventional conditions. On leaving the lyophilizing equipment, dehydrated beads a few millimeters in diameter are obtained. These very easily turn into powder, simple friction causing the very porous structure of the obtained beads to crumble.

1.2 Special experimental conditions

The experimental conditions implemented to cool the coffee samples analyzed in the subsequent examples are as follows:
Conventional freezing in a cold room, grinding and then spreading the pieces on the trays of the lyophilizing equipment; this condition is called “Reference.”
Cryogenics under low pressure (corresponds to a relative pressure equivalent to approximately 0.5 bars obtained by bringing into contact with a cryogenic fluid), to dissolve a small quantity of gas but still benefit from the shape and temperature advantages of the cryogenics method “under pressure,” then directly spreading the obtained beads on the trays of the lyophilizing equipment. This condition is called “Cryo LP,” for Low Pressure.
Cryogenics under 5 bars of pressure, then spreading of the obtained beads directly on the trays of the lyophilizing equipment; this condition is called “Cryo MP,” for Medium Pressure.

1.3 Preparation of coffee used as matrices

For the tests carried out on coffee, the matrix was prepared from a preparation of 3 L of filtered coffee, separated into 3 batches of 1 L each and then subjected to cooling as described above.

### Example 2: Reduction in Method Execution Time

The method according to the present disclosure allows dehydrated products to be prepared in a shorter time than the deep-freezing-lyophilization method of the state of the art.

First of all, the sample preparation time is reduced, since pressurized cryogenics is an instantaneous method, unlike freezing and cryogenics method without applied pressure. The product can then be lyophilized directly, without a prior cooling step, since the temperature of the products is about −60° C. when entering the lyophilizing equipment.

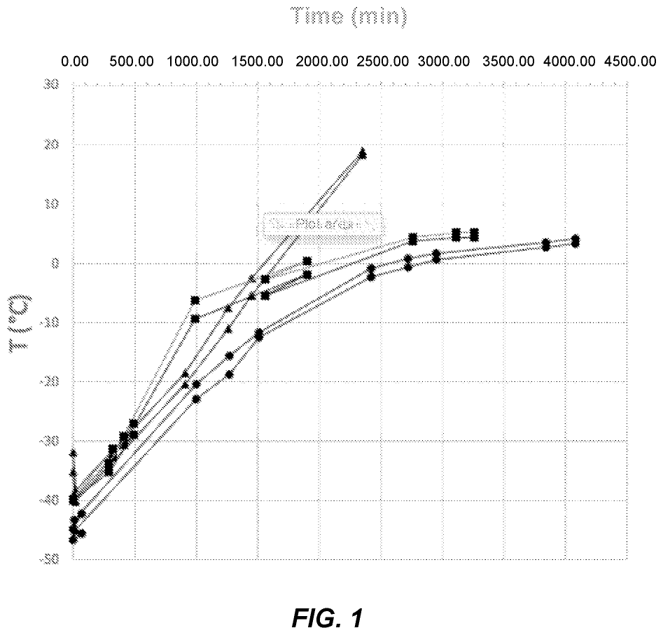

The time required to obtain a lyophilized product was studied. The result is shown in FIG. 1.

These are three coffee samples prepared by applying the particular experimental conditions described in paragraph 1.2.

First of all, it is observed that the time required to extract all the available water is halved when Cryo cryogenics under 5 bars (MP) is used, compared to the use of conventional freezing(Reference). This time savings is considerable. It is also observed that Cryo cryogenics under low pressure (LP) also induces an interesting benefit, although less significant.

### Example 3: Physicochemical Properties of Coffee Powders

The properties of the powders obtained by the method according to the present disclosure were then compared with those of powders obtained by a conventional method (Reference).

3.1 General observations

It is observed that regardless of the pressure applied, the cryogenized beads containing dissolved gas (Cryo LP and Cryo MP) make it possible to obtain a fine powder, which can be handled simply because it can be measured and is not sticky. The resulting powder also does not pick up moisture easily when left under ambient conditions.

Conversely, the Reference lyophilization only makes it possible to obtain product agglomerates, which must generally be reprocessed (by grinding, for example) to facilitate or even allow their use.

3.2 Density of coffee powders

Very surprisingly, the lyophilized powders obtained from cryogenized products containing dissolved gas (Cryo LP and Cryo MP) are much less dense than those obtained from conventionally frozen products.

Table 1 shows the density measurements carried out on the coffee powders obtained under the three different conditions.

TABLE 1
Apparent density of coffee
powders without settling

| | Powder |
|---|---|
| Method | density |
| Reference | 1.15 |
| Cryo LP | 0.78 |
| Cryo MP | 0.40 |

It was observed that the Cryo MP powder is almost 3 times less dense than the Reference powder, which is considerable.

3.3 Observations of particles in microcopy

These measurements could also be correlated with microscopic observations made on the three coffee powders, obtained under the three experimental conditions.

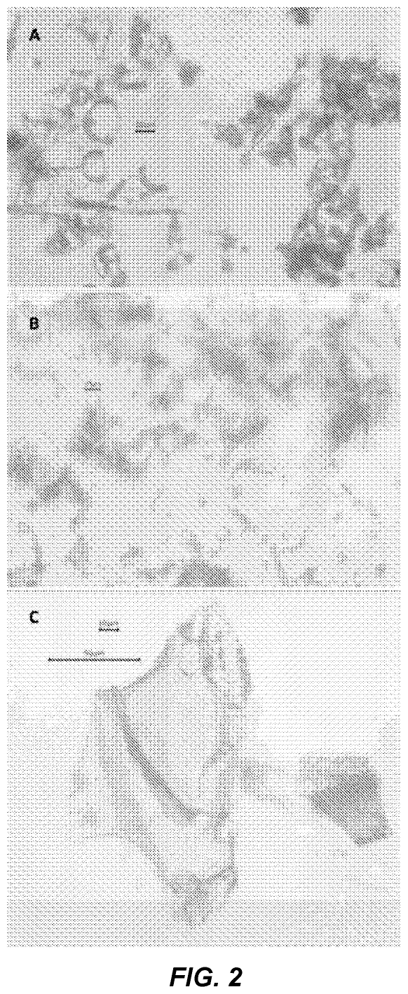

Reproductions of these microscopic observations are shown in FIG. 2.

Surprisingly, although the frozen beads are non-porous (the incorporated gas is dissolved and allows the product to retain its “full,” non-porous structure), a release of nitrogen takes place during lyophilization, which allows the formation of small, very porous particles. The more the quantity of dissolved gas increases, the greater the porosity. The method under pressure thus produces a powder of very low apparent density (see Table 1) that does not require grinding.

The powder can still be easily compacted if necessary.

3.4 Dissolution rate

Observations of particle sizes can be correlated with differences in the rate of dissolution of the obtained coffee powders in water. The hot rehydration being very fast (a few seconds), dissolution kinetics were carried out at 22° C. to be more discriminating.

The rehydration times of preparations at 1% of coffee are shown in Table 2 below. For each preparation condition, 1 g of lyophilized coffee is poured into 99 g of demineralized water stirred using a magnetic stirrer (IKA Lab Disk model set at 160 rpm). The measured rehydration time is that necessary so that no more solid grains are visible in the solution.

TABLE 2
Rehydration in distilled water at 22° C.
of 1 g of coffee in 99 g of water

| | Rehydration |
|---|---|
| Sample | time |
| Reference Coffee | 1′53″ |
| Cryo LP Coffee | 1′23″ |
| Cryo MP Coffee | 58″ |

These results show that the rehydration is almost 2 times faster for the Cryo MP samples compared to that of the Reference samples. This is explained by a lower density of the lyophilized powder, which leads to greater porosity and therefore an accentuated capillary effect allowing faster hydration.

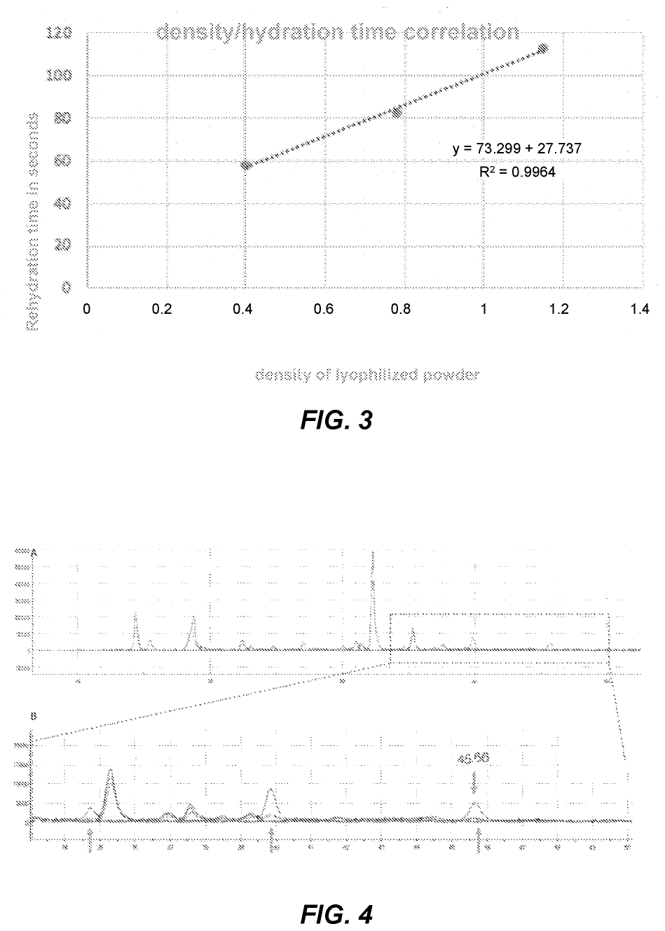

This correlation is shown in FIG. 3.

This correlation was not expected, as very low-density powders generally experience difficulties with rehydration. They tend to float more easily on the surface of the liquid, water in this case, without dissolving there. In the case of the present disclosure, this correlation is most certainly explained by the aspects of crystal size, illustrated in FIG. 2, which in turn induce these variations in apparent densities.

### Example 4: Organoleptic Properties of Powders

The organoleptic properties were then studied.

The olfactory profile of each coffee was determined by double ultra-rapid gas phase chromatography (Heracles II electronic nose, AlphaMos). To do this, 0.01 g of each sample was taken in a 20 ml vial and placed at 40° C. for 1 hour to allow the release of the aromas, which are then analyzed automatically. Each analysis is repeated 3 times.

4.1. Electronic nose analysis

Three analyses (in triplicate) were performed on each sample and the average chromatograms are presented in FIG. 4.

It has been noted that the profiles of the 3 preparations (frozen reference, cryogenized LP and MP) show the same aroma peaks. The LP cryogenic coffee even has 2 additional peaks at retention times of 40 s and 46 s. Overall, it has also been noticed that the intensity of the detected peaks is higher for the products cryogenized under pressure compared to the reference. This results in a better olfactory intensity during the rehydration of the coffee (cf. sensory analysis)

Very surprisingly, the organoleptic properties of the products obtained from cryogenized products containing dissolved gas (Cryo LP (C₀) and Cryo MP (C₅)) under pressure are much less altered than those obtained under Reference conditions from conventionally frozen products (Cref). In particular, the tastes and aromas are much better preserved in the case where cryogenics under pressure (LP or MP) is used.

Two remarkable phenomena are observed. First, the peak intensity is lower for the Reference (FIG. 4A, lower curves), regardless of the peak. The highest intensity is most often obtained for Cryo LP conditions. In FIG. 4B, it is also observed that certain peaks are very pronounced for Cryo LP (peak highlighted by the arrows), while they are very slight or even absent in the other two cases. Analysis of the molecules responsible for these peaks indicates that they are most likely known aromatic molecules of freshly roasted coffee. These molecules are all available from purified flavor molecule suppliers to enhance the taste of coffee and other food preparations. It is therefore very interesting that these molecules are “naturally” more present in the preparations obtained by way of the method that is the subject of the present disclosure.

These results show the greatest aromatic potential of the lyophilized coffees obtained according to the method that is the subject of the present disclosure.

4.2 Principal Component Analysis

A Principal Component Analysis (PCA) is performed on the different coffee samples to assess their overall olfactory rendering by the electronic nose.

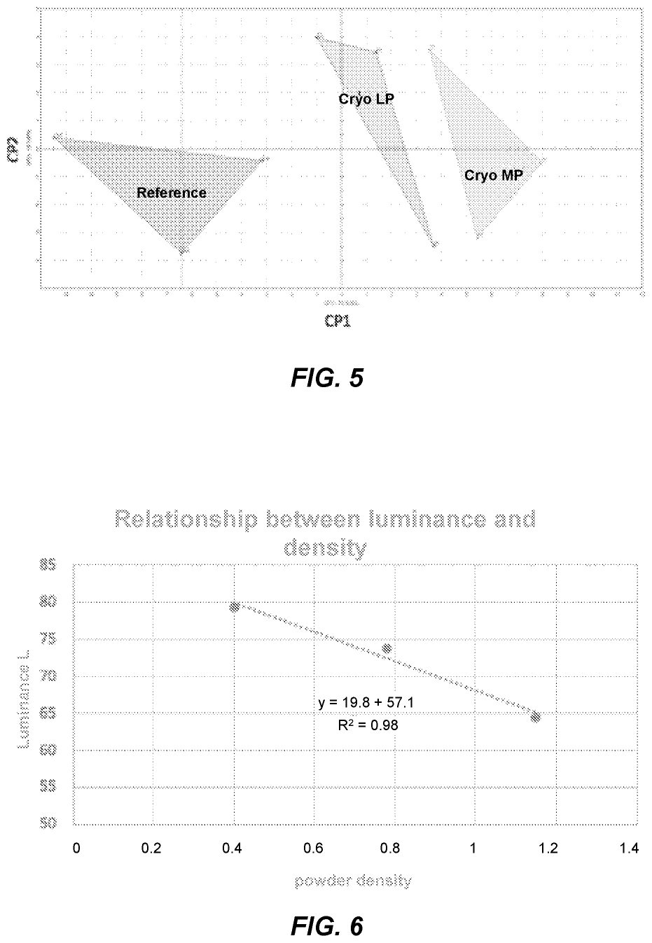

The results are shown in FIG. 5.

This overall analysis leads to obtaining a discrimination index between the samples of 62, which is significant. More than 75% of this discrimination index can be explained by the difference in area between the peaks (represented by the main component 1, denoted CP1).

The Euclidean distances between samples are shown in Table 3.

TABLE 3
Euclidean distance between samples

| | Euclidean | |
|---|---|---|
| Compared Samples | distance | Significance |
| (Cryo LP-Cryo MP) | 4.8 | p <0.001 |
| (Cryo LP-Reference) | 8.84 | p <0.001 |
| (Cryo MP-Reference) | 12.72 | p <0.001 |

Euclidean distances are important between pressurized cryogenic coffees and the frozen Reference. The products are very significantly different. Cryogenics under pressure produces coffees with an aromatic profile that is different from conventional deep freezing. The higher the pressure, the greater the distance. Overall, the increase in pressure increases the olfactory intensity of coffees.

This is a completely surprising result and validates the interest of using cryogenics under pressure.

4.3 Sensory analysis of coffees

45 people were asked to compare 3 coffees prepared according to the methods described above (Reference, Cryo LP and Cryo MP), to detect whether differences exist between the 3 coffees and, if differences exist, to classify them in order of preference.

The “benchmark” coffee was detected as strongly different from the other 2 coffees by 93% of the tasters. This coffee was detected 76% of the time as “less aromatic” than the other two.

The two Cryo LP and Cryo MP coffees were detected as different by 61% of the tasters. This difference is therefore less sensitive than the difference between cryogenic coffees and the Reference. Among the tasters who judged the 2 cryogenic coffees to be different, 64% preferred the Cryo MP coffee because of a “more marked aromatic development.”

This analysis shows that the method described here produces a coffee that is different from a conventional freezing method (Reference) and that increasing the pressure in the method improves the aromatic quality of the coffee.

### Example 5: Powder Colors

The color of the coffee powders was analyzed as a function of the three preparation conditions described above.

The colorimetric analyses were carried out using a DataColor Konica-Minolta colorimeter according to the standardized measurement procedure in the L, a, b reference system.

The results are shown in Table 4.

TABLE 4
Colorimetric analyses of coffee powders
according to the L, a, b standard.

| Method | L | a | b |
|---|---|---|---|
| Cryo MP | 79.25 | 11.14 | 27.07 |
| Cryo LP | 73.75 | 11.55 | 23.65 |
| Reference | 64.42 | 8.23 | 11.83 |

The measurements show that the “luminance L” (also called “clarity”) increases with the pressure exerted during lyophilization and therefore according to the density of the powder. The coffee cryogenized under 5 bars of pressure and then lyophilized has a distinctly lighter shade (higher luminance) than the other samples.

This relationship between density and luminance is validated in FIG. 6, which shows the strong correlation (R²=0.98) that exists between the densities and luminances of 3 lyophilized coffee powders.

For the color factors (a and b), it appears that the “Cryo MP and Cryo LP” methods lead to similar values, while the Reference freezing method provides a less red (parameter a) and less yellow (parameter b) powder than the other two cryogenic powders. The color difference ΔE* between the Reference and the Cryo MP powder is highly significant (ΔE*=21.46). This significant difference in color allows the identification of cryonically pressurized powders.

These results are an additional indicator of the difference in the quality of the coffee powder depending on the lyophilization methods implemented.

In conclusion, coffees prepared under pressure, according to the method of the present disclosure, are clearer, more aromatic, and dissolve better than coffee prepared according to the Reference method.

### Example 6: Preparation of a Custard Flavored with “Cryo MP” Coffee

A lyophilized coffee extract has been used in a culinary preparation.

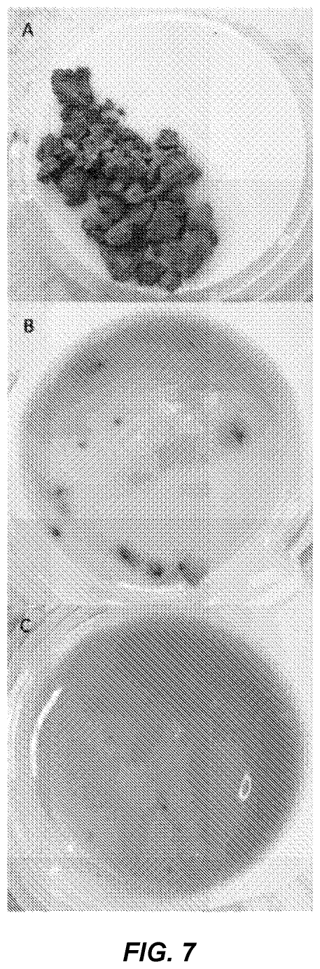

“Cryo MP” coffee was used as a food ingredient to flavor custard. The photos in FIG. 7 describe the 3 stages of dissolving Cryo MP coffee in the cream at 8° C.

In step A, the Cryo MP coffee powder is placed on the custard. The brown powder contrasts with the white cream.

In step B, the Cryo MP coffee is lightly mixed into the custard. There is a slight coloration of the cream

In step C, the appearance of the cream is observed after the coffee has been mixed. The color of the cream is more intense.

The appearance of this food preparation shows that the coffee obtained by the described method can be used as an ingredient and that different products can be obtained depending on the mixing and solubilization time of the “Cryo MP” coffee in the custard.

1. A method of preparing a lyophilized coffee comprising:
a) providing a coffee preparation in the form of a liquid, semi-liquid or pasty matrix;
b) dissolving an inert gas in the matrix by passing the matrix through a zone dense in gas molecules, a density of the gas in the zone being obtained (i) either by the gas flow generated by the evaporation of a cryogenic fluid, (ii) or by an elevation of the pressure, (iii) or by the combination of these two methods (i) and (ii);
c) cryogenizing the gas-rich matrix obtained by the dissolving of the inert gas in the matrix at a pressure allowing the dissolved gas to be maintained to obtain frozen granules, particles or beads;
d) lyophilizing the granules, particles or beads; and
e) obtaining the dehydrated coffee in powder form.

2. The method of claim 1, wherein the inert gas is nitrogen.

3. Lyophilized coffee obtained by the method of claim 1.

4. The coffee of claim 3, wherein the coffee comprises spherical particles.

5. The coffee of claim 3, wherein the coffee comprises particles having a particle size less than 30 microns when measured by optical microscopy.

6. The coffee of claim 3, wherein the coffee comprises compacted particles.

7. A composition comprising a lyophilized coffee as recited in claim 3.

8. The composition of claim 7, comprising a mixture of different lyophilized coffees.

9. The composition of claim 7, further comprising at least one other ingredient, chosen from among milk, chocolate, chicory, flavors and carrier molecules.

10. A method of using coffee according to claim 3 for the preparation of drinks or food or cosmetic preparations.

11. The method of claim 1, wherein the density of the gas in the zone is obtained by the gas flow generated by the evaporation of a cryogenic fluid.

12. The method of claim 1, wherein the density of the gas in the zone is obtained by an elevation of the pressure.

13. The method of claim 1, wherein the density of the gas in the zone is obtained by a combination of the gas flow generated by the evaporation of a cryogenic fluid and an elevation of the pressure.
